(19) United States
(12) Patent Application Publication
(10) Pub. No.: US 2007/0191602 A1
(43) Pub. Date: Aug. 16, 2007

(54) Crystalline form of cefdinir cesium salt

(76) Inventors: Vinod Kumar Kansal, Haryana (IN); Dhirenkumar N. Mistry, Gujarat (IN); Saurabh Pandey, Uttar Pradesh (IN); Rakesh Patel, Gujarat (IN)

Correspondence Address: KENYON & KENYON LLP, ONE BROADWAY, NEW YORK, NY, 10004, US

(21) Appl. No.: 11/593,261

(22) Filed: Oct. 31, 2006

Related U.S. Application Data
(60) Provisional application No. 60/732,098, filed on Oct. 31, 2005.

(51) Int. Cl. C07D 501/14

(52) U.S. Cl. 540/222

(57) ABSTRACT

Provided is the cesium salt of cefdinir, processes for its preparation and its use in the preparation of cefdinir.

### CROSS-REFERENCE TO RELATED APPLICATIONS

This application claims the benefit of priority to U.S. provisional Application Ser. No. 60/732,098, filed Oct. 31, 2005, hereby incorporated by reference.

### FIELD OF THE INVENTION

The present invention encompasses cefdinir cesium salt and the solid state chemistry of it.

### BACKGROUND OF THE INVENTION

Cefdinir is a third generation cephalosporin antibiotic for oral administration and has a broader antibacterial spectrum over general gram positive and gram negative bacteria than other antibiotics for oral administration. Cefdinir, currently marketed as OMNICEF®, is an antibiotic prescribed in a 300 mg oral capsule or a suspension of 125 mg/5 mL. OMNICEF® is prescribed for respiratory and ear infections. Cefdinir is 7-(Z)[2-(2-aminothiazol-4-yl)-2-hydroxyiminoacetimido]-3-vinyl-3-cephem-4-carboxylic acid and has the following structure:

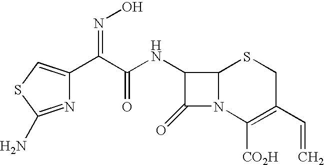

Examples 14 and 16 of U.S. Pat. No. 4,559,334 disclose the synthesis of cefdinir. In examples 14, cefdinir is obtained by reacting benzhydryl 7-(4-bromoacetoacetamido)-3-vinyl-3-cephem-4-carboxylate in dichloromethane and acetic acid with isoamyl nitrite at −3° C. to −5° C., followed by addition of acetylacetone. Thiourea was added and the benzyhydryl group was cleaved with trifluoroacetic acid. After work up, the organic layer was acidified and cooled at 0° C. to obtain the crystalline cefdinir. Compound 9 of example 2 discloses the sodium salt of cefdinir.

U.S. Pat. No. 4,935,507 discloses two methods of obtaining crystalline cefdinir. Crystalline cefdinir may be crystallized from methanol to obtain crystalline cefdinir Form A. Alternatively the Crystalline form is stepwise purified. In the stepwise process, the amorphous form was dissolved in water, washed with saturated sodium bicarbonate, acidified, passed by column chromatography, and treated with activated charcoal. The pH of the resultant solution was adjusted to 1.8 at 35° C. and the resultant crystalline cefdinir Form A was collected. The '507 patent shares one common inventor with the '334 patent and the same assignee. The '507 patent characterizes the product of examples 14 and 16 of the '334 patent as a crystalline like amorphous product, not a crystalline product. The '507 patent further states “the amorphous product has disadvantages that it is bulky, not so pure, unstable and insufficient in filtration rate, therefore it is not suitable for a pharmaceutical product or is not easy to handle in pharmaceutical preparations, in producing it in a large scale or in storage.”

PCT publication WO 98/45299 discloses a cefdinir dicyclohexylamine salt and that cefdinir may be purified via the dicyclohexylamine salt.

According to the PCT publication WO 02/098884, cefdinir is prepared by treating a cefdinir intermediate with a formic acid-sulfuric acid mixture or a formic acid-methanesulfonic acid mixture to obtain a crystalline salt of cefdinir and reacting the crystalline salt with a base in a solvent.

PCT publication WO 03/050124 describes a novel crystalline cefdinir potassium dihydrate, a process for its preparation and its use for the preparation of cefdinir.

US publication US 2004/0242556 discloses a crystalline form of Cefdinir, 7β-[(Z)-2-(2-amino-4-thiazolyl)-2-hydroxyiminoacetamido]-3-vinyl-3-cephem-4-carboxylic acid, named crystal B, a process to prepare it and the use of cefdinir crystal B in pharmaceutical compositions.

Cefdinir compositions often contain a high amount of impurities. For example, the Physician's Desk Reference describes Cefdinir as “white to slightly yellow-brownish solid.” There is a need in the art to prepare cefdinir with a desirable amount of purity on an industrial scale.

### SUMMARY OF THE INVENTION

In one embodiment, the invention encompasses cefdinir cesium.

In another embodiment, the invention encompasses a process for preparing cefdinir cesium of claim 1 comprising combining water (or a mixture of water and a water-miscible organic solvent) cefdinir, a base and a source of cesium ions to obtain cefdinir cesium.

In another embodiment, the invention encompasses a process for preparing cefdinir cesium salt comprising combining cefdinir with water, or a mixture of water and a water-miscible organic solvent, to obtain a reaction mixture, and combining the obtained reaction mixture with a base and a source of cesium ions.

In another embodiment, the invention encompasses a process for preparing cefdinir cesium salt comprising combining 7-amino-3-vinyl-3-cephem-4-carboxylic acid (7-AVNA), an organic solvent selected from the group consisting of: C₃-C₇ ketone, dimethyl formamide, dimethyl acetamide and cyclic ethers, water, and mixtures thereof, and O-acetyl thioester to obtain a reaction mixture; adding an organic base; adding C₁-C₆ halogenated hydrocarbon, C₂-C₆ ester, or C₂-C₈ cyclic ether; and adding a base and a source of cesium ions to obtain a precipitate.

In another embodiment, the invention encompasses a process for preparing cefdinir cesium salt comprising reacting 7-amino-3-vinyl-3-cephem-4-carboxylic acid with O-acetyl thioester in a mixture of tetrahydrofuran and water while stirring, cooling the reaction mixture to about 15-20° C., adding triethylamine to the reaction mixture to obtain a pH of about 8.0 to 8.2, adding methylene chloride to the reaction mixture while maintaining the temperature and stirring, separating the organic and aqueous phases, adding ammonium chloride to the aqueous phase to maintain the pH at about 7.8 to about 8.2, adding cesium carbonate solution the aqueous phase to obtain a precipitate of cefdinir cesium.

In another embodiment, the invention encompasses a crystalline form of cefdinir cesium salt characterized by a powder x-ray diffraction pattern with peaks at 4.7, 9.1, 9.4, 10.7, 12.2, 13.3, 15.9, 16.8, 19.4, 21.5, 22.2, 22.9, 25.8, 26.3, and 27.0±0.2 degrees two-theta.

In another embodiment, the invention encompasses a process for preparing cefdinir comprising converting cefdinir to cefdinir cesium by reaction with a base and a source of cesium, and converting the cefdinir cesium back to cefdinir by reaction with an acid.

In another embodiment, the invention encompasses a process for preparing cefdinir comprising converting cefdinir cesium to cefdinir by reaction with an acid.

In another embodiment, the invention encompasses a process for cefdinir having a purity of more than about 99%.

### BRIEF DESCRIPTION OF THE FIGURES

FIG. 1 illustrates a powder XRD pattern of crystalline form of cefdinir cesium salt prepared according to Example 1.

FIG. 2 illustrates an IR pattern of crystalline form of cefdinir cesium salt prepared according to Example 2.

### DETAILED DESCRIPTION OF THE INVENTION

The present invention provides a process for preparation of cefdinir on industrial scale, using cefdinir cesium salt. The cesium salt described in the present invention allows for obtaining cefdinir with a desirable degree of purity. Furthermore, the cesium salt can be formed in the last step of cefdinir synthesis, where cefdinir forms and precipitates out as the cesium salt. As such, the formation of the cesium salt can be incorporated in an industrial process for preparation of cefdinir.

The present invention provides cefdinir cesium salt having the following structure:

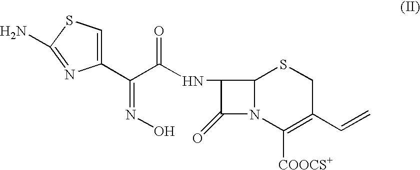

The present invention also provides cefdinir cesium salt with a purity of about above 99%, more preferably, of about above 99.5%, most preferably, of about above 99.8% as measured by HPLC area percent.

The cefdinir cesium salt may be in a crystalline form.

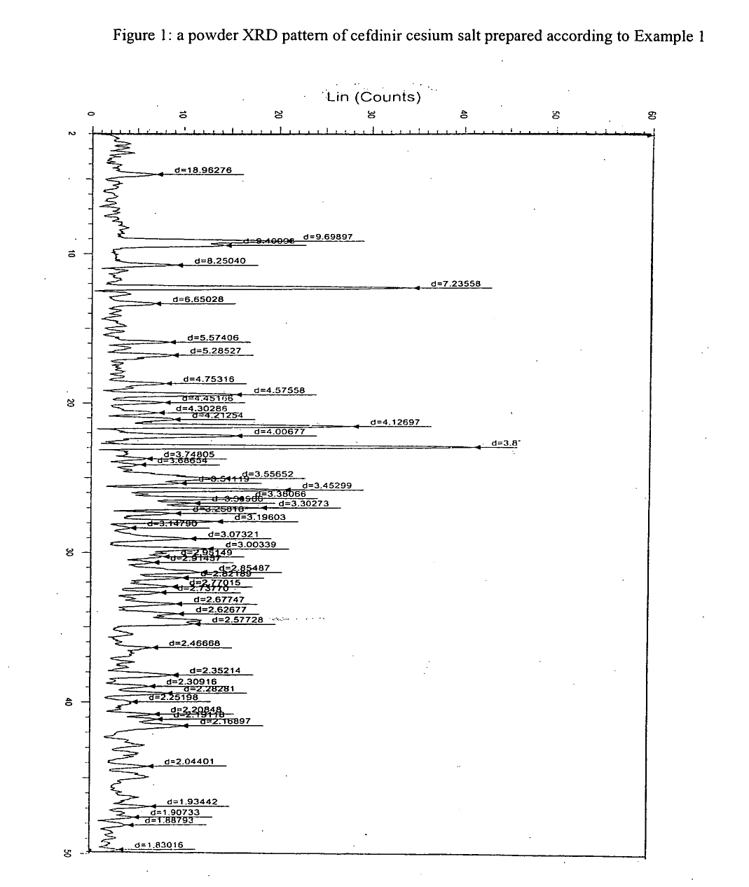

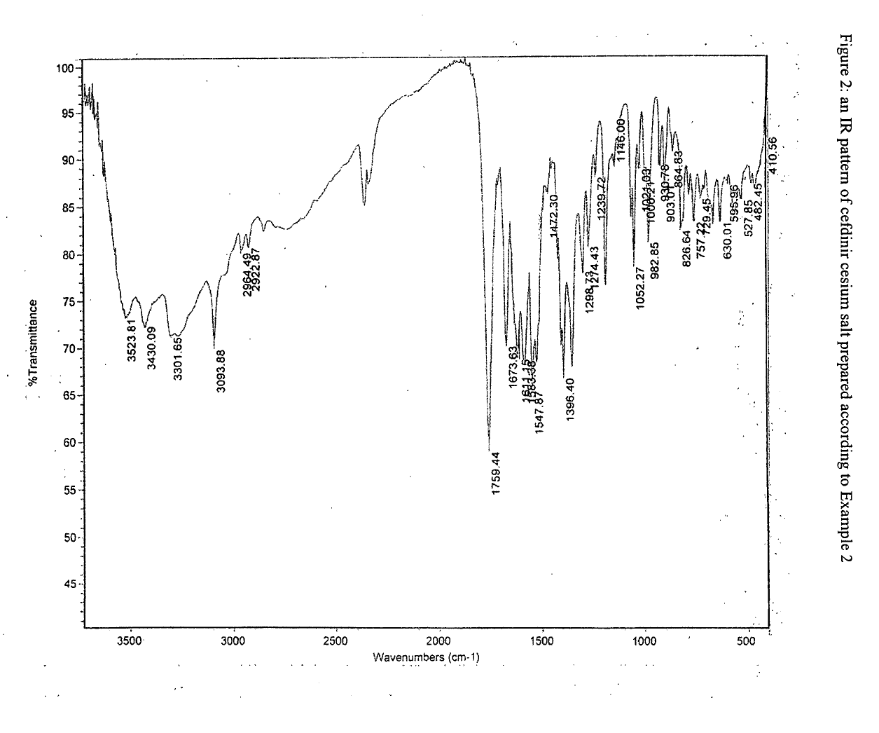

The present invention also provides crystalline cefdinir cesium salt characterized by a X-ray powder diffraction pattern having peaks at about 4.7, 9.1, 12.2, 21.5 and 22.9±0.2 degrees two-theta. The crystalline form may be further characterized by a X-ray powder diffraction pattern having peaks at about 9.4, 10.7, 13.3, 15.9, 16.8, 19.4, 22.2, 25.8, 26.3, and 27.0±0.2 degrees two-theta. The crystalline form may be also substantially identified by the PXRD pattern depicted in FIG. 1. The above crystalline form may be further characterized by an IR spectrum with peaks at: 3523.8, 3430, 3301, 3093.8, 2964.4, 2922.8, 1759.4, 1673.6, 1611.1, 1583.3, 1547.8, 1472.3, 1396.4, 1298.7, 1274.4, 1239.7, 1146, 1052.2, 1021, 1000.2, 982.8, 930.7, 903, 864.8, 824.6, 757.2, 729.4, 630, 595.9, 527.8, 482.4 and 410.5 (cm⁻¹). The crystalline form may be also substantially identified by the IR spectrum depicted in FIG. 2. The crystalline form may have a water content of about 5 to about 22% by weight as measured by Karl Fisher.

The present invention provides a process for preparing cefdinir cesium salt by combining cefdinir with water or a mixture of water and a water-miscible organic solvent to obtain a reaction mixture, and combining the obtained reaction mixture with a base which is also a source of Cesium ions. Preferably, the water-miscible organic solvent is selected from the group consisting of: C₃-C₇ ketone, C₁-C₅ alcohol, alkyl nitrile, acetonitrile and cyclic ethers. Preferably, the C₃-C₇ ketone is acetone. Preferably, the C₁-C₅ alcohol is selected from the group consisting of: isopropyl alcohol, methanol and ethanol. Preferably, the alkyl nitrile is selected from the group consisting of: propyl nitrile and ethyl nitrile. Preferably, the cyclic ether is selected from the group consisting of: dioxane, tetrahydrofuran, and 2-methyl tetrahydrofuran. Most preferably, the solvent is acetone. Preferably, prior to the addition of the base and a source of Cs ions, a suspension is obtained. Preferably, the base which is also a source of Cs ions is selected from the group consisting of: cesium carbonate, cesium bicarbonate and cesium hydroxide.

Impurities that may form during the reaction may be removed by use of active carbon, a chelating agent and a filter. Preferably, the suspension containing cefdinir cesium is cooled. Preferably, the cooling is to a temperature of about 0° C. to about 15° C., more preferably, to a temperature of about 5° C. to about 10° C. The crystals may then be recovered by conventional techniques, such as filtration. The crystals may be further washed and dried. Preferably, the washing is with the same water immiscible solvent used during the process. Preferably, the drying is under atmospheric pressure. Preferably, the obtained cefdinir cesium salt has a purity of about above 98%, more preferably, of about above 99%, most preferably, of about above 99.5%. Preferably, the obtained product is crystalline cefdinir cesium salt characterized by an X-ray powder diffraction pattern with peaks at about 4.7, 9.1, 9.4, 10.7, 12.2, 13.3, 15.9, 16.8, 19.4, 21.5, 22.2, 22.9, 25.8, 26.3, and 27.0±0.2 degrees two-theta.

The present invention provides a process for preparing cefdinir cesium salt by combining 7-amino-3-vinyl-3-cephem-4-carboxylic acid (7-AVNA), an organic solvent selected from the group consisting of: C₃-C₇ ketone, dimethyl formamide, dimethyl acetamide and cyclic ethers, water, and O-acetyl thioester to obtain a reaction mixture; adding an organic base; adding C₁-C₆ halogenated hydrocarbon, C₂-C₆ ester, or C₂-C₈ cyclic ether; and adding a base and a source of Cesium ions to obtain a precipitate. Preferably, the C₃-C₇ ketone is acetone. Preferably, the cyclic ether is tetrahydrofuran. Preferably, prior to the organic base addition, the reaction mixture is cooled. Preferably, the cooling is to a temperature of about 0° C. to about 25° C., more preferably, to a temperature of about 15° C. to about 20° C. Preferably, the base is added to obtain a pH of about 7.8 to about 9, preferably, to obtain a pH of about 8 to about 8.9. Preferably, the base is selected from the group consisting of: triethylamine, tributylamine and N-methyl morpholine. Preferably, after the base addition, additional amount of water is added. Preferably, the C₁-C₆halogenated hydrocarbon is selected from the group consisting of: methylene chloride, chloroform and ethylene dichloride. Preferably, the C₂-C₆ ester is ethyl acetate. Preferably, the C₂-C₈ cyclic ether is tetrahydrofuran. The solvent and the water in the process create a two phase system. Preferably, the aqueous phase and the organic phase are separated. Preferably, the aqueous phase is further extracted with methylene chloride. The base is added to the aqueous phase. Preferably, the base which is also a source of Cs ions is selected from the group consisting of: cesium carbonate, cesium bicarbonate and cesium hydroxide. Preferably, the process further comprises seeding with cefdinir Cs salt. The precipitate may be recovered by conventional techniques, such as filtration. The precipitate may be further washed and dried. Preferably, the washing is with the same water immiscible solvent used during the process. Preferably, the drying is under atmospheric pressure. Preferably, the drying is under atmospheric pressure. Preferably, the obtained cefdinir cesium salt has a purity of about above 98%, more preferably, of about above 99%, most preferably, of about above 99.5%. Preferably, the obtained product is crystalline cefdinir cesium salt characterized by an X-ray powder diffraction pattern having peaks at about 4.7, 9.1, 9.4, 10.7, 12.2, 13.3, 15.9, 16.8, 19.4, 21.5, 22.2, 22.9, 25.8, 26.3, and 27.0±0.2 degrees two-theta.

Preferably, the process for preparing Cefdinir Cs salt comprises reacting 7-amino-3-vinyl-3-cephem-4-carboxylic acid with O-acetyl thioester in a mixture of tetrahydrofuran and water while stirring, cooling the reaction mixture to about 15-20° C., adding triethylamine to the reaction mixture to obtain a pH of about 8.0 to 8.2, adding methylene chloride to the reaction mixture while maintaining the temperature and stirring, adding while maintaining the temperature and stirring, separating the organic and aqueous phases, adding ammonium chloride to the aqueous phase to maintain the pH at about 7.8 to about 8.2, adding cesium carbonate solution the aqueous phase to obtain a precipitate of cefdinir cesium.

The present invention provides a process for preparing cefdinir by preparing Cefdinir Cs salt and then converting it to cefdinir. Preferably, conversion to Cefdinir comprises: dissolving cefdinir Cs salt in water; and adding an acid. Preferably, the solution of the cesium salt is prepared in water. Preferably, the temperature is decreased to about 0° C. to about 15° C. Impurities that may form in the solution may be removed by use of active carbon, a chelating agent and a filter. Preferably, the acid is selected from the group consisting of: hydrochloric acid, adipic acid, oxalic acid and succinic acid and sulfuric acid. More preferably, the acid is sulfuric acid. Preferably, the acid is added to obtain a pH of between about 2.2 to about 2.6. Preferably, when the process is without cooling, the obtained product is cefdinir form A. Form A is characterized by an X-ray powder diffraction pattern having peaks at about 8.8, 11.7, 17.8, 19.2, 21.5, 22.0, 23.4, 24.5, and 27.6±0.2 degrees two-theta. Preferably, when the process includes a cooling step, the obtained product is cefdinir form B. Form A is characterized by an X-ray powder diffraction pattern having peaks at about 5.9, 7.8, 8.0, 11.8, 15.7, 16.2, 18.1, 18.7, 21.0, and 22.3 degrees two-theta. Preferably, the precipitate is filtered and dried. Preferably, the drying is under reduced pressure and a temperature of below about 40° C.

Preferably, the obtained cefdinir has a purity of above 99%, more preferably, a purity of above 99.5%, most preferably, a purity of above 99.8%, as measured by HPLC area percentage.

The present invention also provides cefdinir having a purity of above 99%, more preferably, a purity of above 99.5%, most preferably, a purity of above 99.8%, as measured by HPLC area percentage.

The cefdinir used in the processes of the present invention may be prepared by the process provided in the examples or by using cefdinir made by any suitable process. Cefdinir used as starting material may be obtained by, for example, the processes described in U.S. Pat. Nos. 4,559,334, 4,870,168, 6,093,818, 7,105,659 or as described in WO 92/7840, these references are hereby incorporated by reference.

The present invention also encompasses pharmaceutical formulations comprising cefdinir of the present invention, and pharmaceutically acceptable excipient.

The present invention further encompasses a process for preparing a pharmaceutical formulation comprising combining cefdinir of the present invention with at least one pharmaceutically acceptable excipient.

The present invention further encompasses the use of cefdinir of the present invention for the manufacture of a pharmaceutical composition.

Methods of administration of a pharmaceutical composition of the present invention can be administered in various preparations depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like.

The pharmaceutical compositions are preferably administered orally for treatment or prevention of infections, particularly respiratory and ear infections.

Pharmaceutical compositions of the present invention can optionally be mixed with cefdinir and/or other active ingredients. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. ELDRAGIT®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, cefdinir and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol, and invert sugar may be added to improve the tastep.

Preservatives and chelating agents such as alcohol, sodium-benzoate, butylated hydroxy toluene, butylated hydroxyanisole, and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

When preparing injectable (parenteral) pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages can be conveniently presented in unit dosage form and prepared by any of the methods well-known in the pharmaceutical arts.

While the present invention is described with respect to particular examples and preferred embodiments, it is understood that the present invention is not limited to these examples and embodiments. The present invention, as claimed, therefore includes variations from the particular examples and preferred embodiments described herein, as will be apparent to one of skill in the art.

### EXAMPLES

The X-ray powder diffractions according to FIG. 2, were performed on a Siemens D 5000, Operating at 1.2 KV, Cu tube was used, the scanning parameters were: 2-50 deg. 2theta, step time: 0.5 sec.

FTIR was carried out by Nicolet, AVTAR, 370DTGS, manufactured by Thermoelectron. (IR of the potassium salt was carried out after preparing KBr palate.)

Chromatographic Conditions
Detector: UV 254 nm
Column: Synergi 4μ Fusion-R80 A (C18), 4.6 mm×250 mm, Stainless Steel (or equivalent) Make: Thermo electron corporation
Injection Volume: 15 μl
Flow Rate: 1.0 ml/minute
Column Temperature: 40° C.
Run Time: Gradient Elution see below
Sample Temperature: 5° C.

### Example 1

### Preparation of Cesium 7.beta.-[(Z)-2-(2-Amino-4-Thiazolyl)-2-Hydroxymino Acetamido]-3-Vinyl-3-Cephem-4-Carboxylic Acid. (Cefdinir Cesium Salt)

100 g of 7-amino-3-vinyl-3-cephem-4-carboxylic acid (7-AVNA, 0.4419 mol) was added to 1000 ml of Tetrahydrofuran followed by 180 g of O-acetyl thioester (0.4793 mol,) and 500 ml of water with stirring. Cooled the reaction mass to 15-20° C. To this reaction mixture, 62 ml of triethylamine was added slowly at pH of about.8.0-8.2. Stirring was continued and progress of the reaction was monitored by qualitative HPLC until 7-AVNA was less than 1%. At this stage 1000 ml of methylene dichloride was added and stirred for further 15 minutes at 20-25° C. 250 ml of water was added and stirred the reaction mass for 15 minutes at 20-25° C. Separated the layers and to the aqueous layer, aqueous layer was further extracted by 500 ml methylene chloride. Thereafter, 66 g of ammonium chloride were added to the aqueous part in one lot at 20° C.-25° C. and continued maintain the pH of hydrolysis mass 7.8 to 8.2 by addition of 40% w/v aqueous Cesium carbonate solution. The progress of reaction was monitored by qualitative HPLC till O-acetyl cefdinir was less than 0.5%.after completion of hydrolysis reaction mass the precipitation of crystalline cefdinir cesium salts was observed. If precipitation was not observed after clear solution of reaction mass, the mass was seeded with cefdinir cesium salt and stirred at that point for another one hour and then cooled to 5° C. to 10° C. for another one hour. The slurry was filtered and the obtained product was washed with acetone. The product was dried under atmospheric pressure until moisture content of 7.9% w/w. 146 g of product was obtained with 99.0% purity (by HPLC).

### Example 2

Cefdinir (10 g) was suspended in a mixture of acetone (50 ml) and water (50 ml) at 25 to 30° C. Cesium carbonate (4.1 g) was added to this suspension and stirred for 1-3 hours for complete salt formation. The salt suspension was cooled to 5-10° C. and stirred for 60 minutes. The crystal were filtered, washed with acetone and dried to obtain 7.0 g of the title compound (HPLC purity: 99.5%). The product was dried under atmospheric pressure until moisture content in the range of 5.0 to 8.0% w/w.

### Example 3

### Preparation of Cefdinir from Crystalline Cefdinir Cesium Salt

(a) Preparation of Crystalline Cefdinir Form-A.

Cefdinir Cesium salts (100 gms) obtained from above was dissolved in water (2500 ml) at 25 to 30° C. Active carbon (10 gm) and EDTA (1.0 gm) were added to the resulting solution and mixture was stirred for 15-30 minutes at 25 to 30° C. It was filtered through celite and pH of clear solution was adjusted to 2.2 to 2.5 at 25-30° C. by addition of 10% Hydrochloric acid and stirred at that temperature to obtain crystalline cefdinir Form-A (yield 74 g, HPLC 99.8%).

(b) Preparation of Crystalline Cefdinir Form-B.

Cefdinir Cesium salts (100 gms) obtained from the above example was dissolved in water (2500 ml) at 25 to 30° C. Active carbon (10 gm) and EDTA (1.0 gm) were added to the resulting solution and mixture was stirred for 15-30 minutes at 25 to 30° C. It was filtered through celite and pH of clear solution was adjusted to 2.2 to 2.5 at 8 to 12° C. by addition of 10% Hydrochloric acid and stirred at that temperature to obtain crystalline cefdinir Form-B (yield 74 g, HPLC 99.5%).

What is claimed:

1. Cefdinir cesium.

2. The cefdinir cesium of claim 1, wherein the cefdinir cesium is isolated as a solid.

3. The cefdinir cesium of claim 2, wherein the cefdinir cesium is isolated as a crystal.

4. A process for preparing cefdinir cesium of claim 1 comprising combining water (or a mixture of water and a water-miscible organic solvent) cefdinir, a base and a source of cesium ions to obtain cefdinir cesium.

5. A process for preparing cefdinir cesium salt comprising combining cefdinir with water, or a mixture of water and a water-miscible organic solvent, to obtain a reaction mixture, and combining the obtained reaction mixture with a base and a source of cesium ions.

6. The process of claim 5, wherein the water-miscible organic solvent is selected from the group consisting of: C₃-C₇ ketone, C₁-C₅ alcohol, alkyl nitrile, acetonitrile and cyclic ethers.

7. The process of claim 6, wherein the C₃-C₇ ketone is acetone.

8. The process of claim 6, wherein C₁-C₅ alcohol is selected from the group consisting of: isopropyl alcohol, methanol and ethanol.

9. The process of claim 6, wherein the alkyl nitrile is selected from the group consisting of: propyl nitrile and ethyl nitrile.

10. The process of claim 6, wherein the cyclic ether is selected from the group consisting of: dioxane, tetrahydrofuran, and 2-methyl tetrahydrofuran.

11. The process of claim 6, wherein solvent is acetone.

12. The process of claim 5, wherein the base is also a source of cesium.

13. The process of claim 12, wherein the base is selected from the group consisting of: cesium carbonate, cesium bicarbonate and cesium hydroxide.

14. The process of claim 5, wherein the reaction mixture is a suspension.

15. The process of claim 5, wherein the cesium salt precipitates to form a suspension.

16. The process of claim 15, further comprising cooling the suspension to a temperature of about 0° C. to about 15° C.

17. The process of claim 16, wherein the temperature of about 5° C. to about 10° C.

18. The process of claim 5, further comprising recovering the cefdinir cesium.

19. The process of claim 18, further comprising washing and drying the cefdinir cesium.

20. The process of claim 19, the washing is with the same water immiscible solvent used during the process.

21. The process of claim 19, wherein the drying is under atmospheric pressure.

22. The process of claim 5, wherein the obtained cefdinir cesium salt has a purity of above about 98%.

23. The process of claim 22, wherein the purity is above about 99%.

24. The process of claim 23, wherein the purity is above about 99.5%.

25. The process of claim 5, wherein a crystalline cefdinir cesium salt is obtained that is characterized by X-ray powder diffraction peaks at about 0.4.7, 9.1, 9.4, 10.7, 12.2, 13.3, 15.9, 16.8, 19.4, 21.5, 22.2, 22.9, 25.8, 26.3, and 27.0±0.2 degrees two-theta.

26. A process for preparing cefdinir cesium salt comprising combining 7-amino-3-vinyl-3-cephem-4-carboxylic acid (7-AVNA), an organic solvent selected from the group consisting of: C₃-C₇ ketone, dimethyl formamide, dimethyl acetamide and cyclic ethers, water, and mixtures thereof, and O-acetyl thioester to obtain a reaction mixture; adding an organic base; adding C₁-C₆ halogenated hydrocarbon, C₂-C₆ ester, or C₂-C₈ cyclic ether; and adding a base and a source of cesium ions to obtain a precipitate.

27. The process of claim 26, wherein the C₃-C₇ ketone is acetone.

28. The process of claim 26, wherein the cyclic ether is tetrahydrofuran.

29. The process of claim 26, prior to the organic base addition, the reaction mixture is cooled.

30. The process of claim 29, wherein the cooling is to a temperature of about 0° C. to about 25° C.

31. The process of claim 30, wherein the temperature is of about 15° C. to about 20° C.

32. The process of claim 26, wherein the base is added to obtain a pH of about 7.8 to about 9.

33. The process of claim 32, wherein the pH is of about 8 to about 8.9.

34. The process of claim 26, wherein the base is selected from the group consisting of: triethylamine, tributylamine and N-methyl morpholine.

35. The process of claim 26, wherein, after the base addition, additional amount of water is added.

36. The process of claim 26, wherein the C₁-C₆ halogenated hydrocarbon is selected from the group consisting of: methylene chloride, chloroform and ethylene dichloride.

37. The process of claim 26, wherein the C₂-C₆ ester is ethyl acetate.

38. The process of claim 26, wherein the C₂-C₈ cyclic ether is tetrahydrofuran.

39. The process of claim 26, wherein the solvent and the water in the process create a two phase system.

40. The process of claim 26, wherein the aqueous phase and the organic phase are separated.

41. The process of claim 40, wherein the aqueous phase is further extracted with methylene chloride.

42. The process of claim 26, wherein base is added to the aqueous phase.

43. The process of claim 26, wherein the base, which is also a source of cesium ions, is selected from the group consisting of: cesium carbonate, cesium bicarbonate and cesium hydroxide.

44. The process of claim 26, wherein the process further comprises seeding with cefdinir cesium salt.

45. The process of claim 26, further comprising recovering the precipitate.

46. The process of claim 45, wherein the precipitate is further washed and dried.

47. The process of claim 26, wherein the washing is with the same water immiscible solvent used during the process.

48. The process of claim 26, wherein the drying is under atmospheric pressure.

49. The process of claim 26, wherein the obtained cefdinir cesium salt has a purity of above about 98%.

50. The process of claim 49, wherein the purity is above about 99%.

51. The process of claim 50, wherein the purity is above about 99.5%.

52. The process of claim 26, wherein a crystalline cefdinir cesium salt that is obtained is characterized by X-ray powder diffraction peaks at about 4.7, 9.1, 9.4, 10.7, 12.2, 13.3, 15.9, 16.8, 19.4, 21.5, 22.2, 22.9, 25.8, 26.3, and 27.0±0.2 degrees two-theta.

53. A process for preparing cefdinir cesium salt comprising reacting 7-amino-3-vinyl-3-cephem-4-carboxylic acid with O-acetyl thioester in a mixture of tetrahydrofuran and water while stirring, cooling the reaction mixture to about 15-20° C., adding triethylamine to the reaction mixture to obtain a pH of about 8.0 to 8.2, adding methylene chloride to the reaction mixture while maintaining the temperature and stirring, separating the organic and aqueous phases, adding ammonium chloride to the aqueous phase to maintain the pH at about 7.8 to about 8.2, adding cesium carbonate solution the aqueous phase to obtain a precipitate of cefdinir cesium.

54. A crystalline form of cefdinir cesium salt characterized by a powder x-ray diffraction pattern with peaks at 4.7, 9.1, 9.4, 10.7, 12.2, 13.3, 15.9, 16.8, 19.4, 21.5, 22.2, 22.9, 25.8, 26.3, and 27.0±0.2 degrees two-theta.

55. The crystalline form of claim 54, further identified by a powder XRD pattern with peaks at 9.1, 12.2, 21.5, 22.2, and 22.9±0.2 degrees two-theta.

56. The crystalline form of claim 54, further identified by the pattern XRD pattern depicted in FIG. 1.

57. The crystalline form of claim 54, further identified by the IR spectrum depicted in FIG. 2.

58. The crystalline form of claim 54, further identified by a water content of about 5% to about 22% by weight as measured using Karl Fischer titration.

59. A process for preparing cefdinir comprising converting cefdinir cesium prepared by the process of claim 5 into cefdinir.

60. A process for preparing cefdinir comprising converting cefdinir cesium prepared by the process of claim 26 into cefdinir.

61. A process for preparing cefdinir comprising converting cefdinir cesium to cefdinir by reaction with an acid.

62. The process of claim 61, wherein the cefdinir is prepared by reacting 7-amino-3-vinyl-3-cephem-4-carboxylic acid with O-acetyl thioester.

63. Cefdinir having a purity of more than about 99%.

64. The cefdinir of claim 63, wherein the purity is more than about 99.5%.

65. The cefdinir of claim 64, wherein the purity is more than about 99.8%.
